(19) United States
(12) Patent Application Publication
(10) Pub. No.: US 2025/0348805 A1
(43) Pub. Date: Nov. 13, 2025

(54) INFORMATION PROCESSING METHOD, INFORMATION PROCESSING APPARATUS, AND STIMULATION SYSTEM

(71) Applicant: Panasonic Intellectual Property Management Co., Ltd., Osaka (JP)

(72) Inventor: TAKAMASA ANDO, Osaka (JP)

(21) Appl. No.: 19/277,468

(22) Filed: Jul. 23, 2025

Related U.S. Application Data
(63) Continuation of application No. PCT/JP2024/001456, filed on Jan. 19, 2024.

(30) Foreign Application Priority Data
Feb. 14, 2023 (JP) 2023-020770

(51) Int. Cl. G06Q 10/0631; A61M 21/00; A61M 21/02

(52) U.S. Cl. CPC G06Q 10/063114; A61M 21/02; A61M 2021/0016; A61M 2021/0027; A61M 2021/0044; A61M 2021/005; A61M 2021/0066

(57) ABSTRACT

Provided is an information processing method executed by a computer. The information processing method includes: acquiring first information about a period of time during which a user performs an activity; determining, based on the first information, whether a current time has reached a first point in time or not, the first point in time being earlier than a scheduled end time by a predetermined amount of time, the scheduled end time being a point in time at which the activity is to finish; and giving, when the current time is determined to have reached the first point in time earlier than the scheduled end time by the predetermined amount of time, a first stimulus for heightening a degree of relaxation of the user to the user by controlling a stimulation apparatus.

### BACKGROUND

### 1. Technical Field

The present disclosure relates to an information processing method, etc. that is executed by a computer.

### 2. Description of the Related Art

Japanese Unexamined Patent Application Publication No. 2012-171944 proposes a solution for swiftly cooling down or alleviating a heightened state of arousal or autonomic nervous system tension caused by an activity such as work or study after the completion thereof.

### SUMMARY

However, for example, there is a possibility that time might be wasted for relaxing from a tense state after the completion of an activity like work.

One non-limiting and exemplary embodiment provides an information processing method, etc. that makes it possible to reduce the time taken for relaxing from a tense state after the completion of an activity like work.

An information processing method according to certain one aspect of the present disclosure is an information processing method executed by a computer. The information processing method includes: acquiring first information about a period of time during which a user performs an activity; determining, based on the first information, whether a current time has reached a first point in time or not, the first point in time being earlier than a scheduled end time by a predetermined amount of time, the scheduled end time being a point in time at which the activity is to finish; and giving, when the current time is determined to have reached the first point in time earlier than the scheduled end time by the predetermined amount of time, a first stimulus for heightening a degree of relaxation of the user to the user by controlling a stimulation apparatus.

Comprehensive or specific aspects may be realized in the form of a system, an apparatus, a method, an integrated circuit, a computer program, or a non-transitory computer-readable storage medium such as a CD-ROM, or in any selective combination of a system, an apparatus, a method, an integrated circuit, a computer program, and a storage medium.

An information processing method according to certain one aspect of the present disclosure makes it possible to reduce the time taken for relaxing from a tense state after the completion of an activity.

It should be noted that general or specific embodiments may be implemented as a system, a method, an integrated circuit, a computer program, a storage medium, or any selective combination thereof.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 is a block diagram illustrating an example of the configuration of a stimulation system according to an embodiment;

FIG. 2 is a conceptual diagram illustrating an example of a use scene in an embodiment;

FIG. 3 is a flowchart illustrating an example of the operation of a stimulation system according to an embodiment;

FIG. 4 is a conceptual diagram illustrating a first specific example of schedule information in an embodiment;

FIG. 5 is a conceptual diagram illustrating a second specific example of schedule information in an embodiment;

FIG. 6 is a graph showing a first specific example of changes, as time proceeds, of various kinds of data in an embodiment;

FIG. 7 is a graph showing a second specific example of changes, as time proceeds, of various kinds of data in an embodiment;

FIG. 8 is a graph showing a third specific example of changes, as time proceeds, of various kinds of data in an embodiment;

FIG. 9 is a diagram illustrating an example of a vehicle; and

FIG. 10 is a diagram schematically illustrating an example of a view toward front seats from the position of rear seats illustrated in FIG. 9.

### DETAILED DESCRIPTIONS

If a tense state experienced during an activity like work continues even after the activity like work finishes, fatigue will accumulate. Therefore, the fatigue remains, which might affect the next activity like work. This results in a bad spiral of a drop in work-like activity efficiency, extended working hours, and further fatigue accumulation. This might cause the waste of resources, such as power, for performing the activity like work.

With regard to this issue, Japanese Unexamined Patent Application Publication No. 2012-171944 mentioned above proposes a solution for swiftly cooling down or alleviating a heightened state of arousal or autonomic nervous system tension caused by an activity such as work, study, etc. after the completion thereof.

However, for example, there is a possibility that time might be wasted for relaxing from a tense state after the completion of an activity like work because the relaxing from the tense state starts from a point in time at which the activity like work finishes.

Addressing this issue, an information processing method according to a first example is an information processing method executed by a computer, the method including: acquiring first information about a period of time during which a user performs an activity; determining, based on the first information, whether a current time has reached a first point in time or not, the first point in time being earlier than a scheduled end time by a predetermined amount of time, the scheduled end time being a point in time at which the activity is to finish; and giving, when the current time is determined to have reached the first point in time earlier than the scheduled end time by the predetermined amount of time, a first stimulus for heightening a degree of relaxation of the user to the user by controlling a stimulation apparatus.

This makes it possible to put the user in a relaxed state at the point in time at which the activity finishes, on the basis of the first information about the period of time during which the user performs the activity. Therefore, it is possible to improve the efficiency of the next activity. Thus, it is possible to reduce the waste of resources for performing the activity. Moreover, it is possible to reduce the time taken for putting the user in a relaxed state after the activity finishes.

An information processing method according to a second example may be the information processing method according to the first example that further includes: estimating a degree of performance of the activity that the user performs, wherein the first stimulus is given in a case where the current time is determined to have reached the first point in time and, in addition, where the degree of performance has dropped.

This helps prevent misguiding the user who is concentrating on the activity into a relaxed state. Therefore, it is possible to suppress a decrease in the efficiency of the activity.

An information processing method according to a third example may be the following information processing method: in the information processing method according to the second example, based on an operation log that indicates an input operation via a mouse or a keyboard used by the user, the degree of performance is estimated.

This makes it possible to estimate the degree of performance properly, based on the input operation.

An information processing method according to a fourth example may be the information processing method according to any of the first to third examples that further includes: acquiring biometric information on the user; and estimating, based on the biometric information, a degree of tension of the user, wherein the first stimulus is given in a case where the current time is determined to have reached the first point in time and, in addition, where the degree of tension has dropped.

This makes it possible to estimate a proper degree of tension, based on the biometric information. This helps prevent misguiding the user who is concentrating on the activity into a relaxed state. Therefore, it is possible to suppress a decrease in the efficiency of the activity.

An information processing method according to a fifth example may be the information processing method according to any of the first to fourth examples that further includes: increasing an intensity of the first stimulus as the current time comes close to the scheduled end time.

This makes it possible to bring the user closer to a relaxed state gradually. Therefore, it is possible to end the activity smoothly.

An information processing method according to a sixth example may be the information processing method according to any of the first to fifth examples that further includes: acquiring biometric information on the user; estimating, based on the biometric information, the degree of relaxation; and controlling an intensity of the first stimulus according to the degree of relaxation, wherein the intensity of the first stimulus is increased when a rate of increase in the degree of relaxation is lower than a predetermined rate of increase while the first stimulus is being given.

This makes it possible to bring the user closer to a relaxed state gradually in accordance with the predetermined rate of increase in the degree of relaxation. Therefore, it is possible to end the activity smoothly.

An information processing method according to a seventh example may be the following information processing method: in the information processing method according to any of the first to sixth examples, the first stimulus includes at least one stimulus selected from the group consisting of scent, sound, video, light, heat, and airstream.

This makes it possible to, based on the first stimulus, heighten the degree of relaxation of the user efficiently.

An information processing method according to an eighth example may be the information processing method according to any of the first to seventh examples that further includes: acquiring biometric information on the user; and estimating, based on the biometric information, the degree of relaxation; and giving stimuli each of which heightens the degree of relaxation to the user in respective periods of time before the first point in time, the stimulus given in each one of the periods of time being different from the stimulus given in another one of the periods of time, wherein a stimulus that produces a greatest effect of heightening the degree of relaxation, among the stimuli given to the user in the periods of time, is determined, and the determined stimulus is given as the first stimulus to the user.

This makes it possible to give the stimulus that produces the greatest effect to the user, among the stimuli different from one another. Therefore, it is possible to heighten the degree of relaxation of the user efficiently.

An information processing method according to a ninth example may be an information processing method executed by a computer, the method including: estimating a degree of performance of an activity that a user performs; determining, based on the degree of performance, whether or not the activity is in a closing phase that is before the activity finishes; and giving a first stimulus for heightening a degree of relaxation of the user to the user by controlling a stimulation apparatus in a case where the activity is determined to be in the closing phase that is before the activity finishes.

This makes it possible to, based on the degree of performance, put the user in a relaxed state at the point in time at which the activity finishes. Therefore, it is possible to improve the efficiency of the next activity. Thus, it is possible to reduce the waste of resources for performing the activity. Moreover, it is possible to reduce the time taken for putting the user in a relaxed state after the activity finishes.

An information processing apparatus according to a tenth example may be an information processing apparatus that includes: a determination unit that acquires first information about a period of time during which a user performs an activity, and determines, based on the first information, whether a current time has reached a first point in time or not, the first point in time being earlier than a scheduled end time by a predetermined amount of time, the scheduled end time being a point in time at which the activity is to finish; and a control unit that gives, when the current time is determined to have reached the first point in time earlier than the scheduled end time by the predetermined amount of time, a first stimulus for heightening a degree of relaxation of the user to the user by controlling a stimulation apparatus.

This makes it possible to put the user in a relaxed state at the point in time at which the activity finishes, on the basis of the first information about the period of time during which the user performs the activity. Therefore, it is possible to improve the efficiency of the next activity. Thus, it is possible to reduce the waste of resources for performing the activity. Moreover, it is possible to reduce the time taken for putting the user in a relaxed state after the activity finishes.

A stimulation system according to an eleventh example may be a stimulation system that includes: the information processing apparatus according to the tenth example; and the stimulation apparatus.

This makes it possible to give the first stimulus for heightening the degree of relaxation of the user to the user by using the stimulation apparatus included in the stimulation system, without relying on any other system or the like.

These comprehensive or specific aspects may be realized in the form of a system, an apparatus, a method, an integrated circuit, a computer program, or a non-transitory computer-readable storage medium such as a CD-ROM, or in any selective combination of a system, an apparatus, a method, an integrated circuit, a computer program, and a storage medium.

With reference to the drawings, embodiments will be described below. Note that all of the embodiments to be described below show comprehensive or specific examples. Numerical values, shapes, materials, constituent elements, the arrangement positions and connection forms of the constituent elements, steps, the sequential order of the steps, etc. that will be shown in the embodiments below are just examples, and thus shall not be construed to limit the scope of claims.

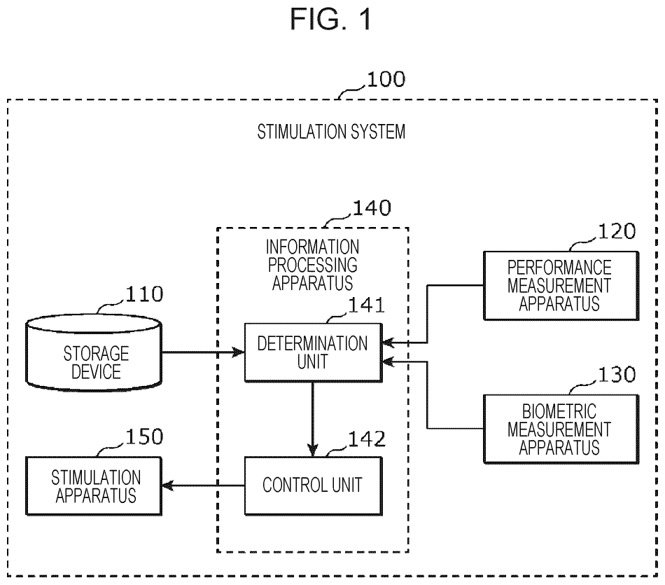

FIG. 1 is a block diagram illustrating an example of the configuration of a stimulation system according to an embodiment. As illustrated in FIG. 1, a stimulation system 100 includes a storage device 110, a performance measurement apparatus 120, a biometric measurement apparatus 130, an information processing apparatus 140, and a stimulation apparatus 150.

The storage device 110 is a device configured to store information. The storage device 110 may be a memory, a storage medium, or the like, or a computer that includes a memory, a storage medium, or the like. The storage device 110 may be included in the information processing apparatus 140 as a storage unit, or may be included in any other apparatus such as a server or the like. The storage device 110 may be connected to a network.

For example, information on the user's schedule is stored in the storage device 110.

The schedule information is an example of information about a period of time during which the user performs an activity like work. The information about a period of time during which the user performs an activity like work may be registered into the storage device 110 as the schedule information by the user.

The schedule information may be inputted by the user into the user's computer, that is, the computer that the user uses for activities, and may be stored in the user's computer, or in a server apparatus connected to the user's computer via a network. The storage device 110 may acquire the schedule information from the user's computer or the server apparatus via the network.

The storage device 110 may acquire identification information such as an ID for identifying the user, and may acquire the user's schedule corresponding to the acquired identification information from a server.

The schedule information may show the user's work-like activity schedule. Alternatively, the schedule information may show a period of time during which a workspace where the stimulation apparatus 150 and the like are installed is to be used. That is, the schedule information may correspond to booth reservation information.

The performance measurement apparatus 120 is an apparatus that measures a degree of the user's work-like activity performance. Measuring the degree of the user's work-like activity performance corresponds to estimating the degree of the user's work-like activity performance. The performance measurement apparatus 120 may be a computer, or a combination of a computer and a sensor.

For example, the performance measurement apparatus 120 may acquire information about a keyboard input by the user who performs an activity like work, or may acquire information about a mouse operation by the user who performs an activity like work. The performance measurement apparatus 120 may acquire information about the voice of the user who performs an activity like work. The performance measurement apparatus 120 may estimate the work-like activity performance on the basis of the acquired information.

For example, in a case where the user attends a meeting as an activity like work, the performance measurement apparatus 120 may acquire information about the voice of the user, and may estimate the work-like activity performance on the basis of the acquired information. For example, the performance measurement apparatus 120 may acquire information about the facial expression of the user, and may estimate the work-like activity performance on the basis of the acquired information. The performance measurement apparatus 120, when doing this, may acquire the information about the facial expression of the user from the biometric measurement apparatus 130 that acquires an image of the face of the user.

The biometric measurement apparatus 130 acquires biometric information on the user who performs an activity like work. The biometric measurement apparatus 130 may be a sensor, or a combination of a computer and a sensor. The biometric information is also called biometric data. For example, the biometric information is used for estimating a degree of the user's tension or a degree of the user's relaxation.

Specifically, the biometric information may indicate fluctuations in heart rate or pulse. For example, the high-frequency component (HF) of fluctuations in heart rate or pulse corresponds to variations in respiration and reflects parasympathetic nervous system activity. The low-frequency component (LF) of fluctuations in heart rate or pulse corresponds to variations in blood pressure and reflects both sympathetic nervous system activity and parasympathetic nervous system activity. Therefore, HF may be used for measuring a degree of activeness of the parasympathetic nervous system, and LF/HF, or LF/(LF+HF), may be used for measuring a degree of activeness of the sympathetic nervous system.

A parameter in time domain, not limited to a parameter in frequency domain, may be used as the biometric information. Specifically, for example, “HR”, which is an average value (beats per one minute) of a total number of heart beats for five minutes, may be used. “Mean” (ms), which is an average value of RR interval for five minutes, may be used. The slower the heart throbs, the greater the RR interval is. Therefore, “Mean” shows a large value when in a relaxed state and shows a small value when in a tense state.

“SDNN” (ms), which is a standard deviation of RR interval for five minutes, may be used. SDNN also shows a large value when in a relaxed state and shows a small value when in a tense state. “RMSSD” (ms), which is the square root of the mean of the squared differences between successive adjacent RR intervals and serves as an index of vagal nerve tension intensity, may be used. RMSSD also shows a large value when in a relaxed state and shows a small value when in a tense state.

“NN50”, which is the total number of successive adjacent RR intervals whose differences therebetween exceeds 50 ms and serves as an index of vagal nerve tension intensity, may be used. “pNN50” (%), which is the percentage of successive adjacent RR intervals whose differences therebetween exceeds 50 ms and serves as an index of vagal nerve tension intensity, may be used. NN50 and pNN50 also show a large value when in a relaxed state and shows a small value when in a tense state.

“SD1”, which is the standard deviation in the vertical axis direction of the Poincaré plot of heart rate variability, may be used. “SD2”, which is the standard deviation in the horizontal axis direction of the Poincaré plot of heart rate variability, may be used.

The biometric information is not limited to fluctuations in heart rate or pulse, and may indicate pupil diameter.

The pupil diameter decreases due to the functioning of the parasympathetic nervous system. That is, a small pupil diameter shows a relaxed state. The biometric information may indicate body motion. Specifically, the biometric information may indicate fluctuations in the position of the head or sways of the center of gravity of the body. For example, stress or anxiety causes vertical wobbling. That is, vertical wobbling in body motion can be a manifestation of a tense state. The biometric information may indicate blood flow, myoelectric potential, eye blinking, facial expressions, or the like.

The biometric measurement apparatus 130 may be a sensor configured to measure the biometric information described above.

The information processing apparatus 140 is an apparatus that processes information. The information processing apparatus 140 may be a computer. In this example, the information processing apparatus 140 includes a determination unit 141 and a control unit 142.

The determination unit 141 is a circuit that processes information. The determination unit 141 may be a processor such as a CPU of a computer. For example, the determination unit 141 determines whether the work-like activity is close to its finish or not on the basis of information obtained from the storage device 110 or on the basis of information obtained from the performance measurement apparatus 120 or the like.

For example, the determination unit 141 may determine whether to give a stimulus to the user or not on the basis of the degree of work-like activity performance. Alternatively, for example, the determination unit 141 may estimate the degree of the user's tension or the degree of the user's relaxation on the basis of the biometric information obtained from the biometric measurement apparatus 130. Then, the determination unit 141 may determine whether to give a stimulus to the user or not on the basis of the degree of tension or the degree of relaxation.

Alternatively, for example, the determination unit 141 may determine whether to give a stimulus to the user or not through “all-things-considered” determination while taking, into consideration, whether the work-like activity is close to its finish or not, the degree of performance, the degree of tension (or the degree of relaxation), and the like.

The determination unit 141 may record the degree of performance, the degree of tension (or the degree of relaxation), and the like into the storage device 110.

The control unit 142 is a circuit that processes information. The control unit 142 may be a processor such as a CPU of a computer.

For example, the control unit 142 controls the operation of the stimulation apparatus 150 in accordance with the result of the determination made by the determination unit 141. Specifically, in a case where the determination unit 141 determines that a stimulus should be given to the user, the control unit 142 transmits a command for causing the stimulation apparatus 150 to output the stimulus. The control unit 142 may transmit a command for adjusting the intensity of the stimulus outputted from the stimulation apparatus 150 on the basis of the degree of tension or the degree of relaxation estimated by the determination unit 141.

The stimulation apparatus 150 is an apparatus that has a configuration for outputting a stimulus, and gives the stimulus to the user by outputting the stimulus. Specifically, the stimulation apparatus 150 outputs a stimulus for heightening the degree of relaxation. The stimulus for heightening the degree of relaxation can be referred to as a relaxing stimulus. More specifically, this stimulus is a stimulus that makes the parasympathetic nervous system more active. This stimulus may be scent, sound, video, light, heat, airstream, or any selective combination thereof, or the like.

For example, an aroma that has a sedative effect may be used as the stimulus. Specifically, cedrol, which is a component of Himalayan cedar, may be used. Cedrol possesses a quite strong sedative effect. Alternatively, Damask rose, a variety of rose, may be used. Damask rose helps regulate the autonomic nervous system, significantly reduces blood flow in the prefrontal cortex, and has a sedative effect. Alternatively, hotrienol, which is an aromatic component included in Darjeeling second flush and has a strong sedative effect, may be used.

Alternatively, lavender may be used. Lavender contains linalyl acetate, which is a component with analgesic properties, produces an effect of reducing the tension of the sympathetic nervous system and making the activity of the parasympathetic nervous system dominant, and is expected to have a sedative effect, too.

Alternatively, chamomile, which is a typical herb that has a relaxing effect, may be used. Chamomile has a great sedative effect and helps alleviate fatigue and tension, etc. caused by stress. Alternatively, clary sage, which is another kind of herb, may be used. Clary sage gives off a sweet muscat-like aroma, has a sedative effect, and induces deep relaxation.

As another example of the stimulus, heat for warming a part of the body may be used. Specifically, heat for warming the lower back may be used. When the intestines get cold, the autonomic nervous system that regulates the function of blood vessels, internal organs, and the like can become imbalanced, causing the sympathetic nervous system to become dominant. Warming the lower back brings the autonomic nervous system back to a balance and causes the parasympathetic nervous system, which promotes sedation and relaxation, to become dominant. Furthermore, warming the lower back can improve blood circulation in the muscles and enhance the function of the stomach and intestines.

Heat for warming the face may be used. For example, warming the face with steam has a relaxing effect on the brain. Heat for warming the soles of the feet may be used. Warming the soles produces a relaxing effect.

As still another example of the stimulus, light such as illumination may be used. Specifically, warm-color illumination may be used. This produces a relaxing effect. A gradual reduction in an amount of light may be used as the stimulus. That is, darkness may be used as the stimulus. This also produces a relaxing effect.

As still another example of the stimulus, sound that produces a relaxing effect may be used. Specifically, a slow heart beat rhythm corresponding to the number of heart beats or so may be used, or an alpha-wave sound corresponding to a healing music may be used.

As still another example of the stimulus, beat sound that produces a relaxing effect may be used.

Specifically, binaural beats may be used. A binaural beat is an auditory illusion that arises in the brain when sounds with different frequencies are presented respectively to the left and right ears. The brain, when listening to sounds with differing frequencies in the left and right ears, compensates for the difference and thus perceives a binaural beat that is not actually being played. For example, when a listener using earphones listens to a 400 Hz sound in the right ear and a 410 Hz sound in the left ear, the brain recognizes the 10 Hz difference. The 10 Hz difference is an alpha-wave sound and produces a relaxing effect.

Alternatively, isochronic tones may also be used. Isochronic tones are rhythmic single tones that play at equal intervals, and are also known as evenly spaced tones. Listening to isochronic tones for a long time makes brain waves calm down, resulting in relaxation.

As still another example of the stimulus, music that produces a relaxing effect may be used. Specifically, classical music or alpha-wave music may be used. Natural sounds such as the sound of ocean waves, flowing rivers, chirping birds, the calls of forest animals, or the like can be used.

As still another example of the stimulus, massage that produces a relaxing effect may be used. Specifically, foot massage may be used, or massage for relaxing stiff shoulder muscles may be used.

As still another example of the stimulus, video that produces a relaxing effect may be used. Specifically, time-lapse video may be used. Time-lapse video is frame-by-frame-advance video that includes a series of still images acquired by performing capturing many times at intervals and played in sequence. To produce a relaxing effect, time-lapse video of rivers, oceans, flowing clouds, or scenes depicting the growth of flowers and plants can be used.

Time-lapse video is a non-limiting example; instead, video of nature scenery may be used. For example, video of vast oceans, expansive grasslands, valleys, forests, sea of clouds, coral reefs, sunrise, sunset, white beaches, underwater scenes, murmuring streams, night skies, Japanese gardens, outer space, or the like can be used.

As still another example of the stimulus, a stream of air that produces a relaxing effect may be used. Specifically, a stream of air that has fluctuations or 1/f fluctuations, or a stream of air that is like a natural breeze of wind, can be used. An aroma used in aromatherapy may be added to the stream of air. Alternatively, a natural aroma of a tree, leaves, or the like may be added to the stream of air.

As still another example of the stimulus, hot air that produces a relaxing effect may be used. Applying hot air to the lower back, the face, the legs, or the like produces a relaxing effect.

The intensity of the stimulus may be, for example, the strength of the scent, the loudness of the sound, the brightness of the video, the intensity of the light, the amount of the heat, the amount of the airstream, or any selective combination thereof, or the like. Basically, the greater the strength of the scent is, the greater the intensity of the stimulus is, and the same relation holds for the loudness of the sound, the brightness of the video, the intensity of the light, the amount of the heat, or the amount of the airstream.

However, in a case where darkness is used as the stimulus, it may be regarded that the intensity of the stimulus increases as the intensity of the light decreases. For example, when a stimulus is given to the user, there occurs an ambient change in the surroundings of the user. The intensity of the stimulus may correspond to the magnitude of the ambient change in the surroundings of the user.

FIG. 1 illustrates just an example of the stimulation system 100. The stimulation system 100 is not limited to the example illustrated in FIG. 1. Not all of the constituent elements illustrated in FIG. 1 do not have to be included in the stimulation system 100. Any one or more of the constituent elements illustrated in FIG. 1 may be included in a system different from the stimulation system 100, or may be omitted from the stimulation system 100.

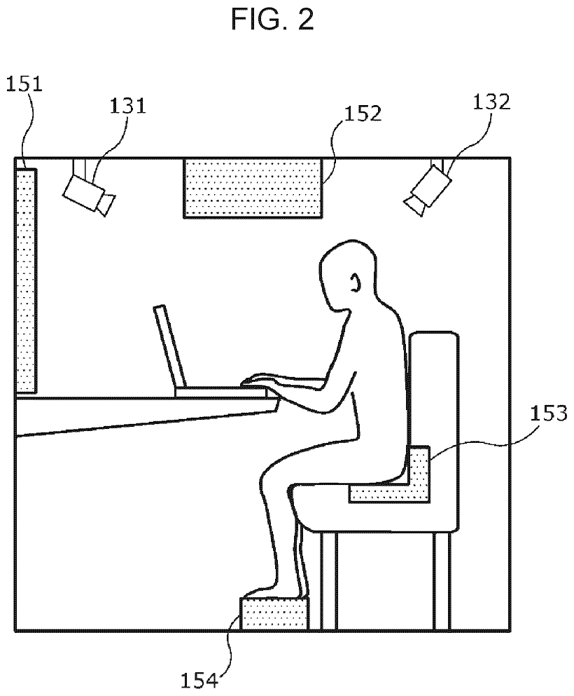

FIG. 2 is a conceptual diagram illustrating an example of a use scene of the stimulation system 100 illustrated in FIG. 1. Biometric measurement apparatuses 131 and 132 and stimulation apparatuses 151 to 154 are illustrated in the example of FIG. 2. Each of the biometric measurement apparatuses 131 and 132 is an example of the biometric measurement apparatus 130 illustrated in FIG. 1. Each of the stimulation apparatuses 151 to 154 is an example of the stimulation apparatus 150 illustrated in FIG. 1.

The biometric measurement apparatus 131 may be equipped with a camera installed ahead of a user who performs an activity like work. The biometric measurement apparatus 131 may generate an image depicting the face of the user by shooting the face of the user. Then, the biometric measurement apparatus 131 may acquire, on the basis of the image, information about the pupil diameter of the user, information about the facial expression of the user, information about the eye blinking of the user, or any selective combination thereof, as biometric information.

The biometric measurement apparatus 131 may, for example, calculate the pulse of the user as the biometric information on the basis of the image. Specifically, the biometric measurement apparatus 131, if equipped with a camera configured to detect light in the wavelength region of visible light, may generate information about the pulse on the basis of light in the wavelength region of green that is obtained from the user.

Alternatively, the biometric measurement apparatus 131, if equipped with a camera configured to detect light in the infrared wavelength region, may generate information about the pulse on the basis of light in the infrared wavelength region that is obtained from the user. In this case, the biometric measurement apparatus 131 may emit infrared light to the user by using a light source configured to perform infrared light emission, and may generate information about the pulse on the basis of the infrared light reflected by the user and coming back from the user.

The biometric measurement apparatus 131 may, for example, be equipped with a camera configured to acquire a change over time of the cerebral blood flow of the user in a non-contact manner, and may acquire information about the change over time of the cerebral blood flow of the user as the biometric information.

The biometric measurement apparatus 132 may be equipped with a camera installed over the user who performs the activity like work. The biometric measurement apparatus 132 generates images corresponding to the movements of the user by shooting the movements of the user. Then, the biometric measurement apparatus 132 may acquire information about the center-of-gravity position of the user and information about the body motion of the user as the biometric information.

The biometric measurement apparatus 130 is not limited to these examples. The biometric measurement apparatus 130 may be an apparatus that is in contact with the user and measures the biometric information of the user. For example, the biometric measurement apparatus 130 may be a wearable device that measures the pulse of the user. Alternatively, the biometric measurement apparatus 130 may be an electrode pad that measures the electrocardiogram of the user as the biometric information.

Alternatively, the biometric measurement apparatus 130 may be a near-infrared spectroscopy (NIRS) apparatus that is worn on the head of the user and measures the cerebral blood flow of the user as the biometric information. Alternatively, the biometric measurement apparatus 130 may be an acceleration sensor that is worn on a part of the body of the user such as the user's wrist.

The biometric measurement apparatus 130 may be a single biometric measurement apparatus or a combination of biometric measurement apparatuses.

The stimulation apparatus 151 may be a display device that displays video as a stimulus. A projector device that projects video as a stimulus may be used in place of the display device that displays video as a stimulus. The stimulation apparatus 152 may be a lighting device that outputs light as a stimulus and illuminates at least a part of a space in which the user performs the activity like work.

Each of the stimulation apparatuses 151 and 152 may be an air-conditioning device that gives a stimulus to the user by blowing air toward the user or by producing a stream of air in the space in which the user performs the activity like work. Such an air-conditioning device may apply hot air to the user. Each of the stimulation apparatuses 151 and 152 may be an aroma diffuser that perfumes, with an aroma as a stimulus, the space in which the user performs the activity like work. Each of the stimulation apparatuses 151 and 152 may be an acoustic device that outputs sound as a stimulus.

Each of the stimulation apparatuses 153 and 154 may be a heater that gives off, as a stimulus, heat for warming the body of the user. Specifically, the stimulation apparatus 153 may be a heater that warms the lower back of the user. The stimulation apparatus 154 may be a heater that warms the soles of the feet of the user.

The stimulation apparatus 150 is not limited to these examples. The stimulation apparatus 150 may apply cold air to the user. The stimulation apparatus 150 may be a single stimulation apparatus or a combination of stimulation apparatuses. The stimulation apparatuses may be controlled in such a way as to operate in a linked manner. For example, sound may be outputted in synchronization with video.

The work-like activity that the user performs may be desk work, a meeting, or the like. The work-like activity that the user performs may be an activity that involves the use of a computer, or may be an activity that does not involve the use of a computer. The work-like activity that the user performs may be work or study. The work-like activity may be referred to as “activity” or “task”, etc.

FIG. 3 is a flowchart illustrating an example of the operation of the stimulation system 100 illustrated in FIG. 1. First, the biometric measurement apparatus 130 measures the biometric information on the user who is performing an activity like work (S101). The measurement of the biometric information continues in subsequent processing, too.

The performance measurement apparatus 120 measures the degree of work-like activity performance by the user who is performing the activity like work (S102). The measurement of the degree of work-like activity performance continues in subsequent processing, too. The performance measurement apparatus 120 may estimate the degree of work-like activity performance on the basis of an operation log that indicates an input operation via a mouse or a keyboard used by the user.

More specifically, the performance measurement apparatus 120 may estimate the degree of work-like activity performance on the basis of the frequency of typing, the amount of typing, and the operation of the mouse. The performance measurement apparatus 120 may estimate that the degree of work-like activity performance is high in a case where the frequency of typing is high, the amount of typing is large, or the operation of the mouse is active.

In an activity that does not involve the use of a computer, the performance measurement apparatus 120 may estimate the degree of work-like activity performance on the basis of the voice, facial expression, body motion, or the like of the user. Specifically, the performance measurement apparatus 120 may estimate the degree of work-like activity performance on the basis of the trends in how often the user speaks during the meeting or how lively the discussion becomes. In an analog activity conducted using a pen and paper, etc., the performance measurement apparatus 120 may estimate the degree of work-like activity performance on the basis of physical sensing data such as hand motion or facial expression, etc.

Next, the determination unit 141 of the information processing apparatus 140 determines whether work-like activity end time is set in the storage device 110 or not (S103). That is, the determination unit 141 determines whether the work-like activity end time is set as the schedule information in the storage device 110 or not.

In a case where the work-like activity end time is set in the storage device 110 (Yes in S103), the determination unit 141 acquires the work-like activity end time from the storage device 110 (S104). Then, in accordance with the work-like activity end time, the determination unit 141 determines whether the work-like activity is close to its finish or not (S105). For example, the determination unit 141 determines whether the current time has reached a point that is earlier than the work-like activity end time by a predetermined amount of time (for example, 10 minutes before it) or not. If the current time has reached the point that is earlier than the work-like activity end time by the predetermined amount of time, the determination unit 141 determines that the work-like activity is close to its finish.

In a case where the work-like activity end time is not set in the storage device 110 (No in S103), the determination unit 141 determines whether the work-like activity is close to its finish or not in accordance with the degree of work-like activity performance (S106). The statement “the work-like activity is close to its finish” corresponds to a situation where the work-like activity is in a closing phase that is before the finishing of the work-like activity. It is anticipated that the degree of work-like activity performance will be on the decline in the closing phase that is before the finishing of the work-like activity. Therefore, the determination unit 141 may determine that the work-like activity is close to its finish in a case where the degree of work-like activity performance exhibits this trend.

Specifically, the determination unit 141 may determine that the work-like activity is close to its finish in a case where predetermined time (for example, three minutes, five minutes, ten minutes, or the like) has elapsed while the frequency of typing per unit time at the present moment is lower than the frequency of typing per unit time at the point in time at which the work-like activity was started. The determination unit 141 may determine whether the work-like activity is close to its finish or not by using information about the work-like activity, such as the amount of mouse movement, body motion, the amount of utterance, or the like, instead of using the frequency of typing.

The determination unit 141 may compare the degree of work-like activity performance at the current time with an average of the degree of work-like activity performance till the current time, instead of comparing the degree of work-like activity performance at the current time with the degree of work-like activity performance at the point in time at which the work-like activity was started. Alternatively, the determination unit 141 may compare the degree of work-like activity performance at the current time with the degree of work-like activity performance at a point that is earlier than the current time by a predetermined amount of time (for example, 15 minutes before it).

If it is determined that the work-like activity is not close to its finish (No in S107), the series of processes for determining whether the work-like activity is close to its finish or not (S103 to S107) is repeated. When the series of processes (S103 to S107) is repeated, once the determination unit 141 acquires the work-like activity end time from the storage device 110 in S104 in the series of processes, the process in S104 does not have to be executed again in the series of processes.

If it is determined that the work-like activity is close to its finish (Yes in S107), the determination unit 141 determines whether the degree of work-like activity performance exhibits a declining trend or not and whether the biometric information exhibits a “shifting-to-relaxed” trend or not (S108). If the degree of work-like activity performance exhibits a declining trend and further if the biometric information exhibits a shifting-to-relaxed trend (Yes in S108), the control unit 142 adjusts the stimulus outputted from the stimulation apparatus 150 and gives it to the user (S109). For example, the stimulus is adjusted in such a way as to guide the user into a relaxed state.

This stimulus is a stimulus that enhances the degree of relaxation, and is different from a stimulus for notifying the user that the work-like activity is close to its finish. For example, this stimulus may be given to the user subliminally. This helps prevent disruption to the progress of the work-like activity.

If the degree of work-like activity performance does not exhibit a declining trend or if the biometric information does not exhibit a shifting-to-relaxed trend (No in S108), the determination of these trends (S108) is repeated, and the processing for giving the stimulus to the user is put on hold. Then, upon determination that the degree of work-like activity performance exhibits a declining trend and further that the biometric information exhibits a shifting-to-relaxed trend, the control unit 142 adjusts the stimulus and gives it to the user.

For example, suppose that a stimulus is given to the user who is performing an activity like work efficiently with deep concentration immediately before finishing this work-like activity; this could be obstructive to the progress of the work-like activity. Therefore, the stimulus is not given to the user if the degree of work-like activity performance does not exhibit a declining trend or if the biometric information does not exhibit a shifting-to-relaxed trend. This helps prevent disruption to the progress of the work-like activity.

After that, the determination unit 141 determines whether the work-like activity has finished or not (S110). If the work-like activity has not finished yet (No in S110), the control unit 142 continues adjusting the stimulus outputted from the stimulation apparatus 150 and giving it to the user (S109). If the work-like activity has finished (Yes in S110), the control unit 142 causes the stimulation apparatus 150 to stop outputting the stimulus.

In the case where the work-like activity end time is set in the storage device 110, the determination unit 141 may determine whether the work-like activity has finished or not on the basis of whether the current time has reached the work-like activity end time or not. In the case where the work-like activity end time is not set in the storage device 110, the determination unit 141 may determine whether the work-like activity has finished or not on the basis of whether predetermined time (for example, ten minutes) has elapsed since determining that the work-like activity is close to its finish.

The operation of the stimulation system 100 is not limited to the example illustrated in FIG. 3. For example, the use of the degree of work-like activity performance is not necessarily needed. In this case, the processing related to the degree of work-like activity performance is omitted. The stimulation system 100 may be configured without the performance measurement apparatus 120. As another example, the use of the biometric information is not necessarily needed. In this case, the processing related to the biometric information is omitted. The stimulation system 100 may be configured without the biometric measurement apparatus 130.

In the trend determination (S108), either one of the two, namely, (a) whether the degree of work-like activity performance exhibits a declining trend or not, or (b) whether the biometric information exhibits a “shifting-to-relaxed” trend or not, may be determined. That is, in S108, the above determination (a) may be performed without the above determination (b), or the above determination (b) may be performed without the above determination (a). The trend determination (S108) may be omitted.

Which one of the work-like activity end time and the degree of work-like activity performance should be used for determining whether the work-like activity is close to its finish or not may have been determined. The determination as to whether the work-like activity end time is set in the storage device 110 or not (S103) may be omitted.

The giving of the stimulus may be continued after the work-like activity finishes. In this case, the determination of the finishing of the work-like activity may be omitted.

If the degree of work-like activity performance does not exhibit a declining trend or if the biometric information does not exhibit a shifting-to-relaxed trend (No in S108), the determination unit 141 may determine whether the work-like activity has finished or not. If the work-like activity has finished, the determination unit 141 may end the repetition of the trend determination (S108). Then, the stimulation system 100 may end the series of operations.

In successive work-like activities, a stimulus may be outputted before each work-like activity finishes. Alternatively, the successive work-like activities may be regarded as a single set of work-like activities, and the stimulus may be outputted before the last one of the successive work-like activities finishes.

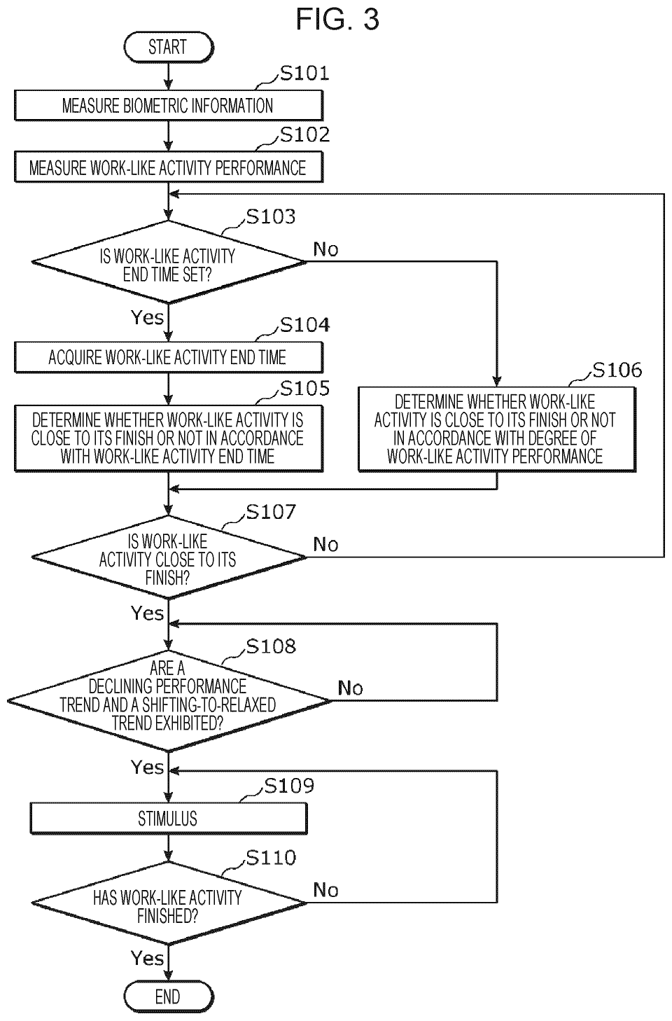

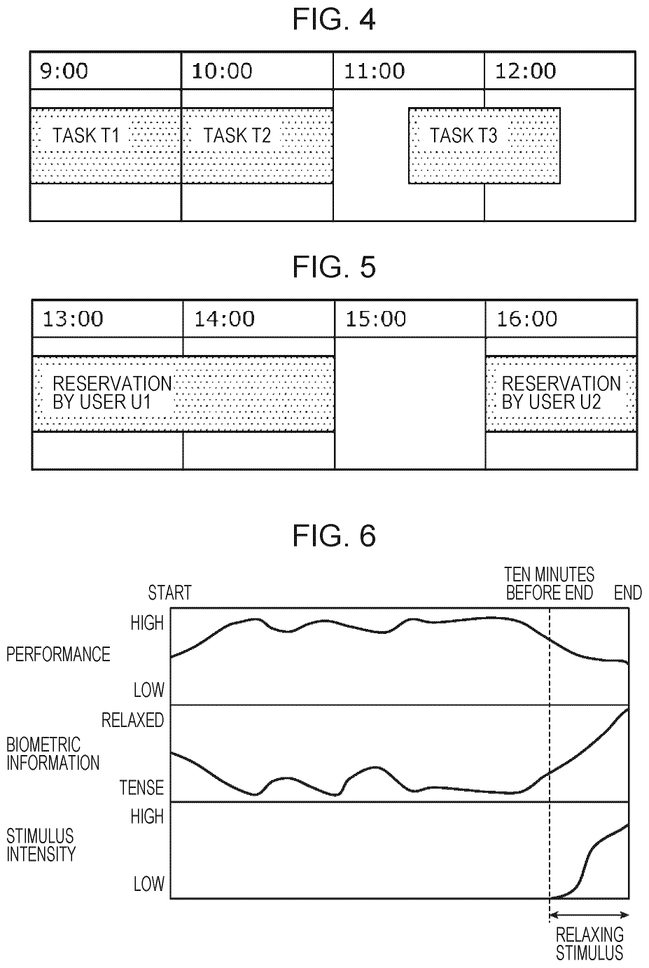

FIG. 4 is a conceptual diagram illustrating a first specific example of the schedule information used in the stimulation system 100 illustrated in FIG. 1. In the example illustrated in FIG. 4, a task T1 and a task T2 are successive timewise.

In this case, it may be determined that the work-like activity is close to its finish when the task T1 is close to its finish, and the stimulus may be outputted. For example, it may be determined that the work-like activity is close to its finish when the current time reaches 9:50, which is ten minutes before the end time of the task T1, and the stimulus may be outputted. It may be further determined that the work-like activity is close to its finish when the task T2 is close to its finish, and the stimulus may be outputted. For example, it may be determined that the work-like activity is close to its finish when the current time reaches 10:50, which is ten minutes before the end time of task T2, and the stimulus may be outputted.

Alternatively, it may be determined that the work-like activity is close to its finish either when the task T1 is close to its finish or when the task T2 is close to its finish, not both, and the stimulus may be outputted. That is, (i) the stimulus may be outputted upon determining that the work-like activity is close to its finish by using the closeness of the task T1 to its finish and not using the closeness of the task T2 to its finish, or (ii) the stimulus may be outputted upon determining that the work-like activity is close to its finish by using the closeness of the task T2 to its finish and not using the closeness of the task T1 to its finish. For example, when the task T1 is close to its finish, the work-like activity is not determined to be close to its finish, and the stimulus is not outputted. When the task T2 is close to its finish, the work-like activity is determined to be close to its finish, and the stimulus is outputted.

The meaning of “the task T1 and the task T2 are successive timewise” in the schedule information is not limited to a case where there is no time interval between the task T1 and the task T2. Even in a case where there is a short task-less time interval (for example, five minutes, ten minutes, or the like) between the task T1 and the task T2, the task T1 and the task T2 may be regarded as being successive timewise.

In the schedule information of the user, information about the location where the user performs the work-like activity may be associated with the schedule of the work-like activity that the user performs. If the schedule information contains information that the task T1, the task T2, and a task T3 are performed at the same location, it may be determined that the work-like activity is close to its finish before the task T3 finishes, without determining that the work-like activity is close to its finish before the task T1 finishes nor before the task T2 finishes.

That is, the schedule information may show more than one task and more than one “worksite” corresponding respectively to said more than one task. It may be determined that the work-like activity is close to its finish when the last task at certain one worksite before a worksite switchover is close to its finish.

FIG. 5 is a conceptual diagram illustrating a second specific example of the schedule information used in the stimulation system 100 illustrated in FIG. 1. As in the example illustrated in FIG. 5, the schedule information may show reservation status of the workspace in which the user performs the work-like activity, without being limited to the direct schedule of the work-like activity that the user performs. In this example, it may be determined that the work-like activity is coming close to its finish when the current time is coming close to 15:00, which is the time at which a user U1 is scheduled to end the use of the workspace.

FIG. 6 is a graph showing a first specific example of changes, as time proceeds, of various kinds of data in the stimulation system 100 illustrated in FIG. 1.

Basically, as in this example, even before the stimulus is outputted, as the work-like activity comes close to its finish, the degree of work-like activity performance decreases, and the biometric information exhibits a shift toward a relaxed state. Then, the outputting of the stimulus starts ten minutes before the work-like activity finishes. This further promotes a shift to a relaxed state.

Starting the outputting of the stimulus ten minutes before the work-like activity finishes is just an example. The timing of starting the outputting of the stimulus does not necessarily have to be ten minutes before the work-like activity finishes.

The intensity of the stimulus may be increased gradually. Specifically, the control unit 142 may increase the intensity of the stimulus as the current time comes close to the scheduled end time. This makes the stimulus less noticeable by the user. Therefore, disruption to the progress of the work-like activity is suppressed.

The outputting of the stimulus may be adjusted in accordance with the biometric information. Specifically, the outputting of the stimulus may be adjusted in such a manner that the degree of relaxation will increase linearly. The outputting of the stimulus may be adjusted in such a manner that, as a result, the intensity of the stimulus will increase non-linearly. For example, the control unit 142 may control the intensity of the stimulus according to the degree of relaxation. The control unit 142 may increase the intensity of the stimulus when the rate of increase in the degree of relaxation is lower than a predetermined rate of increase while the stimulus is being given.

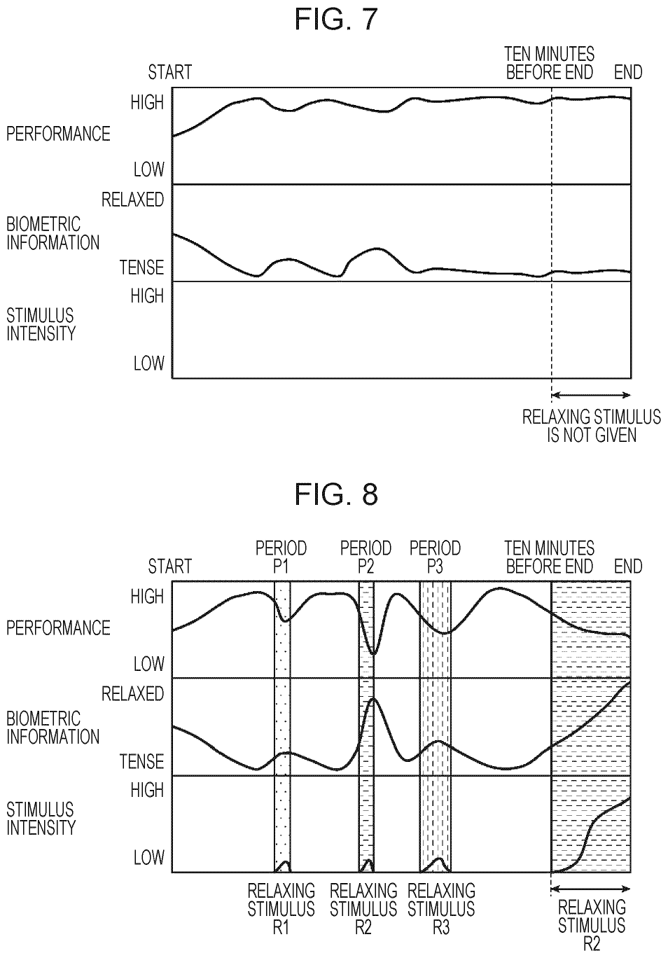

FIG. 7 is a graph showing a second specific example of changes, as time proceeds, of various kinds of data in the stimulation system 100 illustrated in FIG. 1.

For example, sometimes, since the user is performing an activity like work in a concentrated fashion, though the work-like activity is coming close to its finish, the degree of work-like activity performance does not drop, and the biometric information does not exhibit a shift toward a relaxed state. That is, it could happen that a tense state continues though the work-like activity is coming close to its finish. Sometimes the user performs the work-like activity without losing the concentration till the end of the duration of the work-like activity. If the stimulus is outputted in such a case, there is a possibility that it is obstructive to the progress of the work-like activity. To help prevent disruption to the progress of the work-like activity, in such a case, the stimulus is not outputted.

The degree of tension may be measured on the basis of the biometric information. The degree of relaxation may be measured on the basis of the biometric information. Since the degree of tension and the degree of relaxation have a correspondence therebetween, they are mutually permutable.

For example, the degree of relaxation that the biometric information indicates ten minutes before the work-like activity finishes may be compared with the degree of relaxation that the biometric information indicates five minutes further before this ten-minute point in time. Then, it may be determined that the biometric information exhibits a shifting-to-relaxed trend if the result of the comparison indicates that the degree of relaxation that the biometric information indicates ten minutes before the work-like activity finishes is greater.

The degree of relaxation that the biometric information indicates ten minutes before the work-like activity finishes may be compared with the degree of relaxation that the biometric information indicates at the point in time at which the work-like activity was started, or with an average value of the degree of relaxation that the biometric information indicates during the period from the point in time at which the work-like activity was started to the current time. Then, it may be determined that the biometric information exhibits a shifting-to-relaxed trend if the result of the comparison indicates that the degree of relaxation that the biometric information indicates ten minutes before the work-like activity finishes is greater.

It may be determined that the biometric information exhibits a shifting-to-relaxed trend in a case where the degree of relaxation that the biometric information indicates ten minutes before the work-like activity finishes is greater than a middle degree, that is, a middle value between the maximum and the minimum of the index of the degree of relaxation.

With regard to the estimation of the degree of relaxation or the degree of tension, a pseudo task for estimating the degree of relaxation or the degree of tension may be assigned to the user in advance, that is, before the start of the work-like activity. Then, the biometric information such as heart rate (specifically, HF, LF/HF, or the like), pupil diameter, or the like may be measured by means of a sensor (specifically, the biometric measurement apparatus 130). The average value, standard deviation, and the like of the biometric information may be further calculated.

After that, the degree of relaxation or the degree of tension may be estimated, by using the average value and the standard deviation, which have been calculated in advance, from the value of the biometric information measured while the user is performing the work-like activity. Specifically, a value corresponding to the Z score of the user is calculated by subtracting the average value from the measurement value of the biometric information measured while the user is performing the work-like activity and by dividing the result of subtraction by the standard deviation. Then, the calculated value may be used as a standardized degree of relaxation or a standardized degree of tension.

The user to whom the pseudo task is assigned in advance may be different from the user who thereafter performs the work-like activity. The pseudo task may be assigned to more than one user in advance, and the average value and the standard deviation may be calculated for the users.

Alternatively, since the average value and the standard deviation differ from user to user, the user to whom the pseudo task is assigned in advance may be identical to the user who thereafter performs the work-like activity. This makes it possible to estimate the degree of relaxation or the degree of tension that is dependent on the user by using the average value and the standard deviation that are dependent on the user. It is not always needed to calculate the average value and the standard deviation on a work-like-activity-by-activity basis. That is, if the average value and the standard deviation have already been calculated, the average value and the standard deviation having already been calculated may be used.

Multiple kinds of the biometric information are conceivable, such as HF, LF/(LF+HF), pupil size, sways of the center of gravity, cerebral blood flow based on NIRS, myoelectric potential, eye blinking, facial expressions, and the like. For every kind, the Z score may be calculated as the degree of relaxation or the degree of tension by using the average value and the standard deviation from the measurement value. This makes it possible to standardize the degree of relaxation or the degree of tension and to evaluate the degree of relaxation or the degree of tension with the same criterion for evaluation, regardless of the type of the biometric information.

The biometric information may be measured for multiple kinds of the biometric information, and more than one Z score may be calculated from more than one measurement value. Then, the average value of the Z scores may be used as the degree of relaxation or the degree of tension.

Alternatively, from among multiple kinds of the biometric information, one or more kinds effective to the user may be picked, and one or more Z scores may be calculated for the picked one or more kinds thereof. Then, the average value of the one or more Z scores may be used as the degree of relaxation or the degree of tension. Alternatively, the degree of relaxation or the degree of tension may be found using the value of weighted average while weighting the Z scores in accordance with effectiveness to the user, instead of using the average value of the Z scores.

For each of the multiple kinds of the biometric information, whether the kind is effective to the user or not may be determined on the basis of the p value of a significant difference test. The significant difference test may be conducted in measurement performed in advance for calculating the average value and the standard deviation.

The degree of performance may be measured for each of more than one time window obtained by segmentation of time regarding performance measurement.

Specifically, in a case where a keyboard, a mouse, or the like is used, the amount of typing the keyboard, the number of times of clicking the mouse, the amount of scrolling the mouse, or any selective combination thereof in a unit time window, or the like, may be measured as the degree of performance. The degree of performance may be measured according to utterance in the meeting. In this case, the length of voice detection time in a unit time window may be measured as the degree of performance.

The degree of performance may be estimated from facial expressions using machine learning. The degree of performance may be estimated on the basis of how much the eyebrows are lifted in the facial expression, how much the cheeks are lifted therein, how much the corners of the mouth are lifted therein, how much the eyes are open therein, how frequently the eyes blink therein, or any selective combination thereof. The level of concentration evaluated from the facial expression or the like may be estimated as the degree of performance.

FIG. 8 is a graph showing a third specific example of changes, as time proceeds, of various kinds of data in the stimulation system 100 illustrated in FIG. 1.

In this example, even when the work-like activity is not close to its finish, a stimulus for heightening the degree of relaxation is outputted if the degree of work-like activity performance exhibits a declining trend or the biometric information exhibits a shifting-to-relaxed trend. This makes the parasympathetic nervous system more active and promotes refreshing. After that, the stimulus is stopped, and the work-like activity performance recovers.

After the stimulus for heightening the degree of relaxation is outputted, a stimulus that promotes wakefulness may be given to the user. For example, the stimulation apparatus 150 may be capable of outputting the stimulus that promotes wakefulness. Then, the control unit 142 may output the stimulus that promotes wakefulness via the stimulation apparatus 150. The stimulus that promotes wakefulness can be referred to as an awakening stimulus. For example, the stimulus that promotes wakefulness may be a mist, awakening music, intense light, a mint-based fragrance, or the like.

Stimuli different from one another for heightening the degree of relaxation may be outputted, for example, in more than one period of time before the finish while the work-like activity is proceeding toward the finish. In the example illustrated in FIG. 8, a relaxing stimulus R1 is given in a period of time P1, a relaxing stimulus R2 is given in a period of time P2, and a relaxing stimulus R3 is given in a period of time P3. The relaxing stimulus R1, the relaxing stimulus R2, and the relaxing stimulus R3 are stimuli different from one another.

Changes in the degree of relaxation may be recorded, for example, for stimuli given in more than one period of time. For example, the determination unit 141 may record, in the storage device 110, changes in the degree of relaxation estimated according to the biometric information measured by the biometric measurement apparatus 130. Then, when the work-like activity has come close to its finish, among the stimuli, the one that produces the greatest relaxing effect may be outputted.

For example, the stimulus that produces the greatest relaxing effect is a stimulus that causes, after being given, the quickest shift of the biometric information from a tense state to a relaxed state. Alternatively, the stimulus that produces the greatest relaxing effect is a stimulus that has the highest degree of relaxation after being given. Alternatively, the stimulus that produces the greatest relaxing effect is a stimulus whose rate of increase in the degree of relaxation within a certain period of time after being given is the highest.

In the example illustrated in FIG. 8, the relaxing stimulus R2, which produces the greatest relaxing effect among the relaxing stimuli R1 to R3, is outputted.

As explained above, the determination unit 141 acquires first information about a period of time during which a user performs an activity like work, and, based on the first information, determines whether a current time has reached a first point in time or not, the first point in time being earlier than a scheduled end time by a predetermined amount of time, the scheduled end time being a point in time at which the activity like work is to finish. When the current time is determined to have reached the first point in time earlier than the scheduled end time by the predetermined amount of time, the control unit 142 gives a first stimulus for heightening a degree of relaxation of the user to the user by controlling the stimulation apparatus 150.

This makes it possible to put the user in a relaxed state at the point in time at which the activity like work finishes, on the basis of the first information about the period of time during which the user performs the activity like work. Therefore, it is possible to improve the efficiency of the next activity like work. Thus, it is possible to reduce the waste of resources for performing the activity like work. Moreover, it is possible to reduce the time taken for putting the user in a relaxed state after the activity like work finishes.

The performance measurement apparatus 120 may, for example, estimate a degree of performance of the activity like work that the user performs. The control unit 142 may give the first stimulus to the user in a case where the current time is determined to have reached the first point in time and, in addition, where the degree of performance estimated by the performance measurement apparatus 120 has dropped. This helps prevent misguiding the user who is concentrating on the activity like work into a relaxed state. Therefore, it is possible to suppress a decrease in the efficiency of the activity like work.

The case where the degree of performance has dropped may be a case where the degree of performance has become lower than a threshold. The threshold may correspond to the degree of performance a certain time ago, or may correspond to an average degree of performance of the activity like work.

The performance measurement apparatus 120 may, for example, based on an operation log that indicates an input operation via a mouse or a keyboard used by the user, estimate the degree of performance. This makes it possible to estimate the degree of performance properly, based on the input operation.

The biometric measurement apparatus 130 may, for example, acquire biometric information on the user. The determination unit 141 may, based on the biometric information, estimate a degree of tension of the user. The control unit 142 may give the first stimulus to the user in a case where the current time is determined to have reached the first point in time and, in addition, where the degree of tension estimated by the determination unit 141 has dropped.

This makes it possible to estimate a proper degree of tension, based on the biometric information. This helps prevent misguiding the user who is concentrating on the activity like work into a relaxed state. Therefore, it is possible to suppress a decrease in the efficiency of the activity like work. The case where the degree of tension has dropped may be a case where the degree of tension has become lower than a threshold. The threshold may correspond to the degree of tension a certain time ago, or may correspond to an average degree of tension of the activity like work.

The biometric measurement apparatus 130 may, for example, acquire biometric information on the user. The determination unit 141 may, based on the biometric information, estimate a degree of relaxation of the user. The control unit 142 may give the first stimulus to the user in a case where the current time is determined to have reached the first point in time and, in addition, where the degree of relaxation estimated by the determination unit 141 has risen.

This makes it possible to estimate a proper degree of relaxation, based on the biometric information. This helps prevent misguiding the user who is concentrating on the activity like work into a relaxed state. Therefore, it is possible to suppress a decrease in the efficiency of the activity like work. The case where the degree of relaxation has risen may be a case where the degree of relaxation has become higher than a threshold. The threshold may correspond to the degree of relaxation a certain time ago, or may correspond to an average degree of relaxation of the activity like work.

The control unit 142 may, for example, increase an intensity of the first stimulus as the current time comes close to the scheduled end time. This makes it possible to bring the user closer to a relaxed state gradually. Therefore, it is possible to end the work-like activity smoothly.

The biometric measurement apparatus 130 may, for example, acquire biometric information on the user. The determination unit 141 may, based on the biometric information, estimate the degree of relaxation. The control unit 142 may control an intensity of the first stimulus according to the degree of relaxation estimated by the determination unit 141. The control unit 142 may increase the intensity of the first stimulus when a rate of increase in the degree of relaxation estimated by the determination unit 141 is lower than a predetermined rate of increase while the first stimulus is being given.

This makes it possible to bring the user closer to a relaxed state gradually in accordance with the predetermined rate of increase in the degree of relaxation. Therefore, it is possible to end the work-like activity smoothly.

The predetermined rate of increase may be a rate of increase that is defined to be too small to be noticed by the user. For example, the predetermined rate of increase may be zero.

This makes it possible to control the intensity of the stimulus in such a way as, at least, not to decrease the degree of relaxation. The predetermined rate of increase may be determined in accordance with a ratio of a degree of deficiency for reaching a target degree of relaxation (for example, the maximum degree of relaxation) to the time left before the work-like activity end time. This makes it possible to control the intensity of the stimulus in such a manner that the target degree of relaxation will be achieved at the work-like activity end time.

The biometric measurement apparatus 130 may, for example, acquire biometric information on the user. The determination unit 141 may, based on the biometric information, estimate the degree of tension. The control unit 142 may control the intensity of the first stimulus according to the degree of tension estimated by the determination unit 141. The control unit 142 may increase the intensity of the first stimulus when a rate of decrease in the degree of tension estimated by the determination unit 141 is lower than a predetermined rate of decrease while the first stimulus is being given.

This makes it possible to bring the user closer to a relaxed state gradually in accordance with the predetermined rate of decrease in the degree of tension. Therefore, it is possible to end the work-like activity smoothly.

The predetermined rate of decrease may be a rate of decrease that is defined to be too small to be noticed by the user. For example, the predetermined rate of decrease may be zero.

This makes it possible to control the intensity of the stimulus in such a way as, at least, not to increase the degree of tension. The predetermined rate of decrease may be determined in accordance with a ratio of a degree of excess for reaching a target degree of tension (for example, the minimum degree of tension) to the time left before the work-like activity end time. This makes it possible to control the intensity of the stimulus in such a manner that the target degree of tension will be achieved at the work-like activity end time.

The first stimulus may, for example, include at least one stimulus selected from the group consisting of scent, sound, video, light, heat, and airstream. This makes it possible to, based on the first stimulus, heighten the degree of relaxation of the user efficiently.

The biometric measurement apparatus 130 may, for example, acquire biometric information on the user. The determination unit 141 may, based on the biometric information, estimate the degree of relaxation. The control unit 142 may give stimuli each of which heightens the degree of relaxation to the user in respective periods of time before the first point in time, the stimulus given in each one of the periods of time being different from the stimulus given in another one of the periods of time.

Then, the control unit 142 may determine a stimulus that produces the greatest effect of heightening the degree of relaxation, among the stimuli given to the user in the periods of time, and may give the determined stimulus as the first stimulus to the user.

This makes it possible to give the stimulus that produces the greatest effect to the user, among the stimuli different from one another. Therefore, it is possible to heighten the degree of relaxation of the user efficiently.

The biometric measurement apparatus 130 may, for example, acquire biometric information on the user. The determination unit 141 may, based on the biometric information, estimate the degree of tension. The control unit 142 may give stimuli each of which lowers the degree of tension to the user in respective periods of time before the first point in time, the stimulus given in each one of the periods of time being different from the stimulus given in another one of the periods of time.

Then, the control unit 142 may determine a stimulus that produces the greatest effect of lowering the degree of tension, among the stimuli given to the user in the periods of time, and may give the determined stimulus as the first stimulus to the user.

This makes it possible to give the stimulus that produces the greatest effect to the user, among the stimuli different from one another. Therefore, it is possible to lower the degree of tension of the user efficiently.

The performance measurement apparatus 120 may, for example, estimate a degree of performance of an activity like work that a user performs. The determination unit 141 may, based on the degree of performance, determine whether the activity like work is in a closing phase that is before the activity like work finishes or not. Then, the control unit 142 may give a first stimulus for heightening a degree of relaxation of the user to the user by controlling the stimulation apparatus 150 in a case where the activity like work is determined to be in the closing phase that is before the activity like work finishes.

This makes it possible to, based on the degree of performance, put the user in a relaxed state at the point in time at which the activity like work finishes. Therefore, it is possible to improve the efficiency of the next activity like work. Thus, it is possible to reduce the waste of resources for performing the activity like work. Moreover, it is possible to reduce the time taken for putting the user in a relaxed state after the activity like work finishes.

The information processing apparatus 140 includes, for example, the determination unit 141 and the control unit 142. The information processing apparatus 140 may further include the stimulation apparatus 150 as a stimulation unit, the performance measurement apparatus 120 as a performance measurement unit, and/or the biometric measurement apparatus 130 as a biometric measurement unit. The information processing apparatus 140 may include, as a storage unit, the storage device 110 for storing first information about the period of time during which the user performs the activity like work.

The stimulation system 100 includes, for example, the information processing apparatus 140 and the stimulation apparatus 150. The stimulation system 100 may further include the performance measurement apparatus 120 and/or the biometric measurement apparatus 130.

Aspects of an information processing apparatus have been described above in accordance with the embodiments; however, the aspects of the information processing apparatus are not limited to the embodiments. Variations which those skilled in the art can think of may be applied to the embodiments, and/or any combination of the constituent elements disclosed in the embodiments may be adopted.

For example, processing executed by a particular constituent element in the embodiments may be executed by another constituent element that takes the place of the particular constituent element. The sequential order of processes may be changed. Processes may be executed concurrently. The ordinal numbers such as the first, second, etc. assigned for the purpose of explanation may be renumbered, removed, or assigned anew. These ordinal numbers do not necessarily correspond to a meaningful order, and may be used for identification among elements.

An information processing method that includes steps performed by the constituent elements of an information processing apparatus may be executed by any system or any apparatus. That is, the information processing method may be executed by the information processing apparatus described above, or by any other system or apparatus.

For example, a part or the whole of the information processing method may be executed by a computer that includes a processor, a memory, an input/output circuit, and the like. In this case, the information processing method may be executed by running, by the computer, a program for causing the computer to execute the information processing method.

For example, the program described above causes a computer to execute an information processing method comprising: acquiring first information about a period of time during which a user performs an activity like work; determining, based on the first information, whether a current time has reached a first point in time or not, the first point in time being earlier than a scheduled end time by a predetermined amount of time, the scheduled end time being a point in time at which the activity like work is to finish; and giving, when the current time is determined to have reached the first point in time earlier than the scheduled end time by the predetermined amount of time, a first stimulus for heightening a degree of relaxation of the user to the user by controlling a stimulation apparatus.

Alternatively, the program described above may cause a computer to execute an information processing method comprising: estimating a degree of performance of an activity like work that a user performs; determining, based on the degree of performance, whether the activity like work is in a closing phase that is before the activity like work finishes or not; and giving a first stimulus for heightening a degree of relaxation of the user to the user by controlling a stimulation apparatus in a case where the activity like work is determined to be in the closing phase that is before the activity like work finishes.

The program described above may be stored in a non-transitory computer-readable storage medium such as a CD-ROM.

Each constituent element of the information processing apparatus may be configured as dedicated hardware, general-purpose hardware that executes the program described above and the like, or a combination of them. The general-purpose hardware may be configured as a memory that stores a program, a general-purpose processor that reads the program out of the memory and executes the program, and the like. The memory may be a semiconductor memory, a hard disk, or the like. The general-purpose processor may be a CPU or the like.

The dedicated hardware may be configured as a memory, a dedicated processor, and the like. For example, a dedicated processor may execute the information processing method described above while referring to a memory.

Each constituent element of the information processing apparatus may be an electric circuit. These electric circuits may constitute a single electric circuit as a whole, or may be electric circuits independent of one another. These electric circuits may correspond to dedicated hardware, or general-purpose hardware that executes the program described above.

### (Miscellaneous 1)

A workspace in which a user performs an activity may be an internal space of a movable body.

During a period of time in which the user performs the activity, the movable body may be stopped or moving.

For example, the movable body may be a vehicle.

The information processing apparatus 140 may be an electronic control unit (ECU) which the vehicle is equipped with as standard equipment, or may be configured separately from an ECU.

Information on the user's schedule may be stored in a storage device installed in the vehicle. The storage device 110 may acquire the schedule information from the user's computer or a server apparatus via a network. The stimulation apparatus 150 may be an air-conditioning device which the vehicle is equipped with as standard equipment. The stimulation apparatus 150 may be a lighting device which the vehicle is equipped with as standard equipment. The stimulation apparatus 150 may be speakers which the vehicle is equipped with as standard equipment.

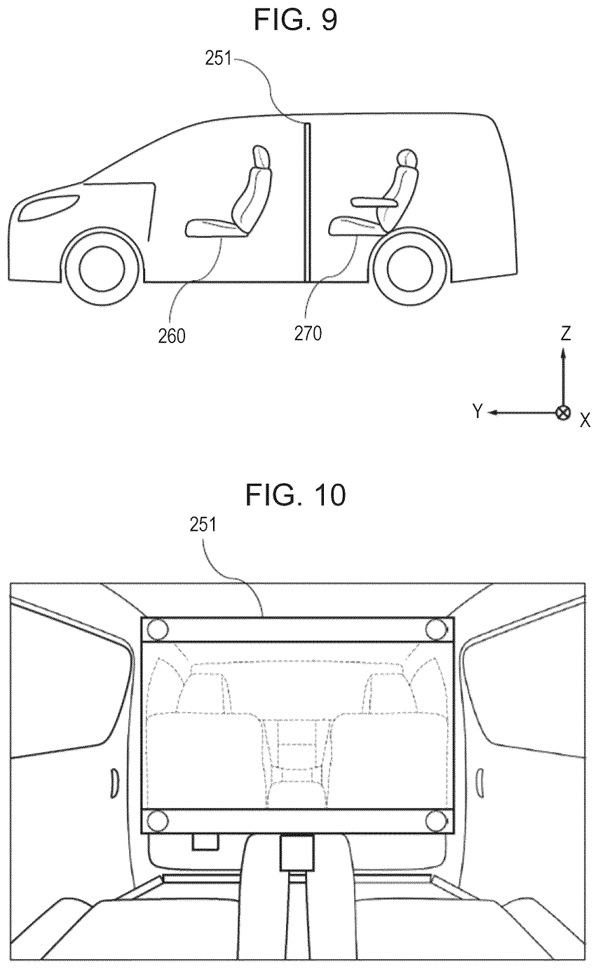

FIG. 9 is a diagram illustrating an example of a vehicle. As illustrated in FIG. 9, a stimulation apparatus 251 may be a display device disposed in the vehicle. The display device may be disposed between the vehicle's front seats 260 and the vehicle's rear seats 270. The front seats 260 include a driver seat and a front passenger seat.

FIG. 10 is a diagram schematically illustrating an example of a view toward the front seats 260 from the position of the rear seats 270 illustrated in FIG. 9. The display device may include a dimming film the transmission factor of which is controllable. A stimulation system may be configured such that the user can see the traveling direction of the vehicle from the rear seats 270 through the display device by controlling the transmission factor.

The display device may have a back surface and a display surface, on the latter of which an image is to be displayed. The display surface may be located at the rear-seat side. The back surface may be located at the front-seat side. That is, the display device may be disposed in such a way as to present an image to the user who performs an activity using a computer, a meeting, etc. on the rear seat.

The display device may be a device that includes a screen and a projector configured to project light onto the screen. The display device may be a monitor that includes pixels and displays video by emitting light from each of the pixels.

The stimulation apparatus 150 may be a seat heater provided in the seat of the vehicle.

Even when the workspace in which the user performs the activity is an internal space of a movable body, the stimulation system 100 may perform the same operations as those illustrated in the flowchart of FIG. 3.

The stimulation system 100 may give the first stimulus to the user and may turn the lighting device off after the end of the period of time during which the activity like work is performed. The stimulation system 100 may give the first stimulus to the user and may turn the display device off after the end of the period of time during which the activity like work is performed. That is, the stimulation system 100 may give the first stimulus to the user and may perform control to decrease the illuminance of the internal space after the end of the period of time during which the activity like work is performed. This makes it possible to guide the user into sleep, with the user's parasympathetic nervous system dominant, at the point in time of completion of the activity. Therefore, the user can fall asleep in a short time after the activity finishes.

The lighting device may be the stimulation apparatus 150, or may be provided separately from the stimulation apparatus 150.

The embodiments of the present disclosure may be modified as in the following variation examples.

### ADDITIONAL REMARK 1

An information processing method executed by a computer, the information processing method comprising:
acquiring first information about a period of time during which a user located inside a movable body performs an activity;
determining, based on the first information, whether a current time has reached a first point in time or not, the first point in time being earlier than a scheduled end time by a predetermined amount of time, the scheduled end time being a point in time at which the activity is to finish; and
giving, when the current time is determined to have reached the first point in time earlier than the scheduled end time by the predetermined amount of time, a first stimulus for heightening a degree of relaxation of the user to the user by controlling a stimulation apparatus disposed inside the movable body.

### ADDITIONAL REMARK 2

The movable body is a vehicle, and
the stimulation apparatus includes one or more apparatuses configured to stimulate the user who sits on a rear seat of the vehicle.

### ADDITIONAL REMARK 3

The stimulation apparatus is a display device,
the display device is disposed between a front seat and a rear seat of the vehicle, and
a display surface of the display device is located at the rear-seat side.

### ADDITIONAL REMARK 4

The information processing method further comprises:
determining, based on the first information, whether the current time has reached the scheduled end time of the activity or not; and
decreasing an illuminance of a space inside the vehicle by controlling the stimulation apparatus when the current time is determined to have reached the scheduled end time of the activity.

The present disclosure can be used as an information processing method for reducing the time taken for relaxing from a tense state after the completion of an activity like work, and can be applied to a stimulation system that alleviates the tense state by means of a stimulus that heightens a degree of relaxation, etc.

What is claimed is:

1. An information processing method executed by a computer, the information processing method comprising:
acquiring first information about a period of time during which a user performs an activity;
determining, based on the first information, whether a current time has reached a first point in time or not, the first point in time being earlier than a scheduled end time by a predetermined amount of time, the scheduled end time being a point in time at which the activity is to finish; and
giving, when the current time is determined to have reached the first point in time earlier than the scheduled end time by the predetermined amount of time, a first stimulus for heightening a degree of relaxation of the user to the user by controlling a stimulation apparatus.

2. The information processing method according to claim 1, further comprising:
estimating a degree of performance of the activity that the user performs, wherein
the first stimulus is given in a case where the current time is determined to have reached the first point in time and, in addition, where the degree of performance has dropped.

3. The information processing method according to claim 2, wherein
based on an operation log that indicates an input operation via a mouse or a keyboard used by the user, the degree of performance is estimated.

4. The information processing method according to claim 1, further comprising:
acquiring biometric information on the user; and
estimating, based on the biometric information, a degree of tension of the user, wherein
the first stimulus is given in a case where the current time is determined to have reached the first point in time and, in addition, where the degree of tension has dropped.

5. The information processing method according to claim 1, further comprising:
increasing an intensity of the first stimulus as the current time comes close to the scheduled end time.

6. The information processing method according to claim 1, further comprising:
acquiring biometric information on the user;
estimating, based on the biometric information, the degree of relaxation; and
controlling an intensity of the first stimulus according to the degree of relaxation, wherein
the intensity of the first stimulus is increased when a rate of increase in the degree of relaxation is lower than a predetermined rate of increase while the first stimulus is being given.

7. The information processing method according to claim 1, wherein
the first stimulus includes at least one stimulus selected from the group consisting of scent, sound, video, light, heat, and airstream.

8. The information processing method according to claim 1, further comprising:
acquiring biometric information on the user;
estimating, based on the biometric information, the degree of relaxation; and
giving stimuli each of which heightens the degree of relaxation to the user in respective periods of time before the first point in time, the stimulus given in each one of the periods of time being different from the stimulus given in another one of the periods of time, wherein
a stimulus that produces a greatest effect of heightening the degree of relaxation, among the stimuli given to the user in the periods of time, is determined, and the determined stimulus is given as the first stimulus to the user.

9. An information processing method executed by a computer, the information processing method comprising:
estimating a degree of performance of an activity that a user performs;
determining, based on the degree of performance, whether or not the activity is in a closing phase that is before the activity finishes; and
giving a first stimulus for heightening a degree of relaxation of the user to the user by controlling a stimulation apparatus in a case where the activity is determined to be in the closing phase that is before the activity finishes.

10. An information processing apparatus comprising:
a determination unit that acquires first information about a period of time during which a user performs an activity, and determines, based on the first information, whether a current time has reached a first point in time or not, the first point in time being earlier than a scheduled end time by a predetermined amount of time, the scheduled end time being a point in time at which the activity is to finish; and
a control unit that gives, when the current time is determined to have reached the first point in time earlier than the scheduled end time by the predetermined amount of time, a first stimulus for heightening a degree of relaxation of the user to the user by controlling a stimulation apparatus.

11. A stimulation system comprising:
the information processing apparatus according to claim 10; and
the stimulation apparatus.
